(19) United States
(12) Patent Application Publication
(10) Pub. No.: US 2003/0028040 A1
(43) Pub. Date: Feb. 6, 2003

(54) Process for producing epoxides from alkenes

(76) Inventors: Johann Seeba, Leverkusen (DE); Anton-Joseph Nagy, Amsterdam (NL); Stephan Volkening, Koln (DE); Georg Wiessmeier, Bergisch Gladbach (DE)

Correspondence Address: BAYER CORPORATION, PATENT DEPARTMENT, 100 BAYER ROAD, PITTSBURGH, PA, 15205, US

(21) Appl. No.: 10/209,262

(22) Filed: Jul. 31, 2002

(30) Foreign Application Priority Data
Aug. 2, 2001 (DE) 10137783.5

(51) Int. Cl. C07D 301/06

(52) U.S. Cl. 549/532

(57) ABSTRACT

The present invention relates to a process for producing epoxides. This process comprises the oxidation of hydrocarbons in the presence of oxygen and at least one reducing agent and a catalyst, characterized in that the reaction mixture is passed through at least one catalyst-containing layer and through at least one adsorbent-containing layer, which adsorbs the epoxide. The catalyst-containing layers and the adsorbent-containing layers are arranged alternately one behind the other such that the reaction mixtures passes through a catalyst-containing layer, and an adsorbent-containing layer.

### BACKGROUND OF THE INVENTION

The present invention relates to a process for producing epoxides by the direct oxidation of hydrocarbons, preferably alkenes, with oxygen in the gas phase, in the presence of at least one reducing agent and a catalyst. The critical feature of the invention is that the reaction mixture is passed through at least one catalyst-containing layer and through at least one adsorbent-containing layer, in which the epoxide is adsorbed, with the catalyst-containing layers and the adsorbent-containing layers being arranged alternately, one behind the other.

The direct oxidation of ethylene to ethylene oxide by molecular oxygen is well known, and is used commercially for the production of ethylene oxide in the gas phase. The typical catalyst for the direct oxidation process contains metallic or ionic silver, which is optionally further modified with various promoters and activators. Most of these catalysts contain a porous, inert catalyst support with small surface areas, such as, for example, alpha-aluminium oxide, to which silver and promoters have been applied. A survey of the direction oxidation of ethylene in the presence of supported silver catalysts was compiled by Sachtler et al. in Catalysis Reviews: Science and Engineering, 23 (1&2), 127-149 (1981).

U.S. Pat. No. 5,623,090 discloses a process for the gas-phase direct oxidation of propylene to propylene oxide with relatively small propylene conversion rates (0.5-1% propylene conversion referred to a 10% propylene feed concentration), but propylene oxide selectivities of >90% with oxygen as oxidizing agent. This involved a gold-titanium dioxidecatalyzed gas phase oxidation with molecular oxygen in the presence of hydrogen at temperatures of 40-70° C. The catalyst used was a commercial crystalline titanium oxide with predominantly anatase modification (P 25, Degussa; 70% anatase and 30% rutile).

Also known are catalysts in which gold particles are applied to a support consisting of dispersed titanium oxide centers on a pure inorganic silicon matrix. These are disclosed in, for example, WO-98/00415-A1, WO-98/00414-A1, WO-99/43431-A1 and EP-A1-0 827 779.

In addition to the relatively low propylene conversion rates, the above-mentioned processes have the great disadvantage that the disclosed catalysts deactivate strongly over time. Typical half-life periods, at standard pressure and 50□C,range from 30 to 150 minutes. Increases in the temperature and/or pressure in an effort to increase the conversion rate, further reduce the half-life periods. This is to be attributed to a subsequent reaction of the products obtained (propylene oxide and water) with the catalyst (formation of glycols). The so-called active centers of the catalyst that are essential for the reaction are covered with secondary products, and are thus not available for further conversions of propylene to propylene oxide.

A development of a process in which the deactivation of the catalysts is prevented or at least strongly suppressed is therefore desirable.

Processes for the oxidation of alkylenes with oxygen or air with the use of suitable catalysts and adsorbents for obtaining corresponding epoxides are also already known and described in, for example, EP-A1-0 372 972, EP-A1-0 328 280, EP-A1-0 336 592, U.S. Pat. No. 4,990,632, EP-A1-0 467 538 and EP-A1-0 646 558. In these references, however, instead of the epoxide being removed by adsorption, the alkylene stream is purified of impurities such as reaction products (e.g. CO, CO₂ or lower hydrocarbons). The resultant purified alkylene stream may then be recycled, and used for the reaction again.

U.S. Pat. No. 5,117,012 discloses the adsorption of epoxylbutadiene in the process of the oxidation of butadiene.

### SUMMARY OF THE INVENTION

An object of the present invention was to provide an improved process for producing epoxides from alkylenes in the presence of oxygen and a reducing agent, wherein the catalyst deactivation is reduced and the yield of epoxides was increased.

A process for producing epoxides by the oxidation of alkylenes in the presence of oxygen and a reducing agent and a catalyst has now been found, which ensures low product concentrations in the gas space and on the catalyst by the adsorption of product. This reduces the deactivation of the catalyst and increases the yield of epoxide.

The object is achieved by a process for the production of epoxides comprising oxidizingone or more hydrocarbons in the presence of oxygen, at least one reducing agent, and a catalyst, and passing the reaction mixture through at least one catalyst-containing layer and then through at least one adsorbent-containing layer, in which the epoxide is adsorbed. In accordance with the present invention, if there is more than one catalyst-containing layer or more than one adsorbent-containing layer, the catalyst-containing layer or layers and the adsorbent-containing layer or layers are arranged alternately, one behind the other, such that the reaction mixture passes through a catalyst-containing layer, then through an adsorbent-containing layer.

### DETAILED DESCRIPTION OF THE INVENTION

As used herein, the phrase “behind” or “one behind the other” is meant here always further to the rear in the flow direction.

If there is only one catalyst containing layer it is a preferred embodiment of the invention that the reaction mixture after leaving the layer is partly led back to pass through the layer again.

As used herein, the term “hydrocarbon” is understood to include unsaturated or saturated hydrocarbons such as, for example, olefins or alkanes, which may also contain hetero atoms such as, for example, N, O, P, S or halogen atoms. The organic component which is to be oxidized may be acyclic, monocylic, bicyclic or polycyclic, and may be monoolefinic, diolefinic or polyolefinic.

In the case of hydrocarbons having two or more double bonds, the double bonds present may be conjugated and non-conjugated. The hydrocarbons from which oxidation products are preferably formed are those hydrocarbons that yield oxidation products whose partial pressure is sufficiently low so as to enable permanent removal of the product from the catalyst. Preferred hydrocarbons include unsaturated and saturated hydrocarbons having 2 to 20 carbon atoms, preferably from 2 to 12 carbon atoms. Most preferably, these include compounds such as, for example, ethylene, ethane, propylene, propane, isobutane, isobutylene, butene-1, butene-2, cisbutene-2, transbutene-2, 1,3-butadiene, pentenes, pentane, 1-hexene, hexenes, hexane, hexadiene, cyclohexene, benzene, etc.

In accordance with the present invention, the process may contain a large number of catalyst-containing layers, preferably from 2 to 20, and most preferably from 3 to 10 catalyst-containing layers, and a large number of adsorbent-containing layers, preferably from 2 to 20, and most preferably from 3 to 10 adsorbent-containing layers. As described above, these layers are arranged in an alternating manner, one behind the other.

The catalyst-containing layers and the adsorbent-containing layers may be arranged in one or more reactors such as, for example one or more serially connected reactors.

In a preferred embodiment of the process of the invention, there are arranged behind each catalyst-containing reactor a plurality of, preferably 2 to 10, and most preferably 2 to 5, adsorbent-containing reactors arranged in parallel, which may be used alternately for adsorption and desorption of the reaction product. Preferably there is one last catalyst-containing reactor after which there are no adsorbent-containing reactors.

A feature of the present invention consists in the fact that in each catalyst-containing layer the alkylene is only partly converted. Preferably, only from 0.01 to 90%, more preferably from 1 to 50%, and most from preferably 2 to 30% of the maximum possible alkylene conversion, is converted in each catalyst-containing layer. It is thereby achieved that the reaction product is discharged out of the reaction mixture in the adsorbent-containing layer which the reaction mixture flows through next, and the selectivity improvement and prolongation of the service life of the catalyst may thereby occur. The maximum possible conversion may be predetermined by, for example, the thermodynamic equilibrium.

The oxygen suitable for the present invention may be used in a wide variety of forms such as, for example, molecular oxygen, air and/or nitrogen oxide. Molecular oxygen is preferred.

Suitable reducing agents for the present invention include those compounds which may take up an oxygen atom, or release a hydrogen atom. Examples of such compounds include compounds such as hydrogen, carbon monoxide or synthesis gas. Hydrogen and carbon monoxide are preferred reducing agents.

Any known source of hydrogen may be utilized in the present invention. Some examples include pure hydrogen, cracker hydrogen, synthesis gas or hydrogen from the dehydrogenation of hydrocarbons and alcohols. In another embodiment of the present invention, the hydrogen may also be produced in situ in a reactor connected upstream by, for example, the dehydrogenation of propane or isobutane, or alcohols such as isobutanol. The hydrogen may also be introduced into the reaction system as a complex-bonded species such as, for example, a catalyst-hydrogen complex.

In addition to the essentially necessary starting material gases described above, optional use may also be made of a diluent gas such as, for example, nitrogen, helium, argon, methane, carbon dioxide, or similar, for the most part inertly behaving gases. Mixtures of the inert components described may also be used. The addition of an inert component is often beneficial for the transport of the heat that is liberated during the exothermic oxidation reaction, and for safety purposes. If the process according to the invention is carried out in the gas phase, it is preferred to use gaseous dilution components such as, for example, nitrogen, helium, argon, methane and optionally, water vapor and carbon dioxide. Water vapor and carbon dioxide are, admittedly, not completely inert, but frequently have a positive effect in small concentrations (<2 vol. % of the total gas stream).

The relative molar ratios of hydrocarbon, oxygen, reducing agent (in particular hydrogen), and optionally, a diluent gas are variable within wide limits.

Oxygen is preferably used in an amount of up to 30 mol %, preferably within the range of 1 to 30 mol %, most preferably of 5 to 25 mol % (based on the total gas stream).

It is preferred to use an excess of hydrocarbon, with respect to the amount of oxygen used (on a molar basis). The hydrocarbon content is typically greater than 1 mol % and less than 96 mol % (based on the total number of moles of the total gas stream. Preferred hydrocarbon contents in the range of from 5 to 90 mol %, and most preferably of from 20 to 85 mol %, are used. The molar reducing agent portion (in particular hydrogen portion), based on the total number of moles of hydrocarbon, oxygen, reducing agent and diluent gas, may be varied within a wide range. Typical reducing agent contents are greater than 0.1 mol %, preferably from 2 to 80 mol %, and most preferably from 3 to 70 mol %.

The catalyst used in the catalyst-containing layer in the process of the present invention preferably comprises a metal catalyst on an oxidic support. Suitable metals to be used as the metal catalyst include the elements cobalt, ruthenium, iridium, nickel, palladium, platinum, copper, silver and gold, with gold being preferred. Combinations of said precious metals are also possible. The oxides of metallic and semi-metallic elements are suitable as support materials. The supports may also consist of oxides of different metallic or semi-metallic elements. Preferred support materials are, for example, titanium oxide and mixtures of titanium oxides and silicon oxides. Such catalysts are described in, for example, DE-A1-199 59 525, DE-A1-100 23 717, the disclosures of which are herein incorporated by reference.

The catalyst-containing layer may also contain other catalysts on a small scale or inert components for dilution of the catalyst.

The oxidation reaction is advantageously carried out at increased reaction pressures. Reaction pressures of greater than 1 bar are preferred, and from 2 to 50 bar are particularly preferred.

The catalyst loading may be varied within wide limits. Preferably catalyst loads in the range of from 0.5 to 100 l gas (total gas stream, i.e. reaction mixture) per ml of catalyst and hour are used, and particularly preferably catalyst loads of from 2 to 50 l gas per ml of catalyst and hour are selected.

During the catalytic oxidation of hydrocarbons in the presence of hydrogen, water is obtained, as a rule, as a companion product to the corresponding selective oxidation product.

The continuous separation of the partial oxidation products from the reaction mixture, which are obtained during the direct oxidation in the presence of oxygen and a reducing agent, is surprisingly also possible in the presence of water and/or water vapor and acidly reacting by-products, by selective adsorption on suitable adsorbents without decomposition of the adsorption products.

Therefore, suitable adsorbents are considered to include all solids which are capable of adsorbing partially oxidized hydrocarbons without decomposition, even in the presence of water and/or water vapor and acidly reacting by-products. The adsorbent-containing layer must at the same time not initiate any secondary reactions of the adsorbed partial oxidation products.

Suitable examples of adsorbents include, for example, zeolites. Hydrophobic zeolites are preferably used as adsorbents in the present invention. Particularly preferred are zeolites of the Faujasit (HY) type with a low aluminium content (e.g. Wessalith DAY or DAZ F20 from Degussa). It is also possible, however, to use molecular sieves or other substances on which the epoxide may preferably be adsorbed, and from which it may be removed again undecomposed, by desorption or washing.

The process according to the invention serves in particular for the production of epoxides from the corresponding alkenes. Preferred alkenes are ethylene, propylene and butylene (e.g. butene-1, butene-2), with propylene being particularly preferred.

Depending on the choice of catalyst used, the process may be carried out at temperatures in the range of from 20 to 400° C., preferably of from 20 to 200° C. At temperatures of more than about 220° C., large amounts of carbon dioxide are formed in addition to the partial oxidation products.

An advantage of the process according to the invention is the increase in the yield of product by the use of adsorbent-containing layers. The adsorbent lowers the epoxide concentration in the gas space and on the catalyst, and thereby reduces the deactivation of the catalyst. Thus, the yield of epoxide is increased.

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

### Example 1

Production of the Above-mentioned Catalyst: Au/TiO₂:

In order to suspend 10 g of titanium oxide hydrate (BET surface of 380 m²/g, 12% water) in 0.31 of deionised water, 100 mg of H(AuCl₄)×H₂O, dissolved in 100 ml of deionised water, were added dropwise at room temperature with stirring within 60 min. In order to precipitate the gold hydroxide, the pH value was adjusted to 8 with a 0.5 molar Na₂CO₃ solution; the pale yellow suspension discolored. The suspension was stirred for 3 h at room temperature, the solid separated and washed 4 times with 25 ml of demineralised water. For drying, the solid was held for 2 h at 150° C. and then for 1 h at 200° C., and the dried catalyst was then calcined in air for 2 h at 250° C. and for 5 h at 400° C.

A catalyst with 0.5 wt % of gold was obtained. Characterisation with TEM (transition electron microscopy) produced nano-scale gold particles with mean particle diameters of approx. 1-6 nm.

### Example 2

Comparison of the Process According to the Invention with the Prior Art Using the Catalyst of Example 1:

Two metal tube reactors of 10 mm inner bore and 20 cm long were used, the temperature of which was controlled by means of an oil thermostat. The reactors were supplied with reaction (i.e. starting material) gases by a set of four mass flow controllers (hydrocarbon, oxygen, hydrogen, nitrogen). For the reaction, two reactors were each filled with 500 mg of a catalyst consisting of 0.5% metallic gold in the form of small particles (2 to 10 nm) supported on a titanium dioxide(Al 5585) support, and 2 g of zeolite (Wessalith DAZ F20 from Degussa) as an adsorbent. In the first reactor, the catalyst was introduced in one layer, followed by one layer of zeolite (comparison example). In the second reactor the amount of catalyst and zeolite was divided into 4 parts of equal size and introduced in the form of alternating layers of catalyst/zeolite (example according to the present invention). The reactors were brought to a temperature of 50° C.

The starting material gases were added to the reactor from above.

The standard catalyst load was 3 l of gas/(g cat. * h). Propylene was selected as “standard hydrocarbon”. A gas stream enriched with nitrogen of the following composition was selected for the carrying out of the oxidation reactions: N₂/H₂/O₂/C₃H₆=14/75/5/6. The amounts are given in vol. % under standard conditions. The reaction gases were analyzed quantitatively by gas chromatography. The gas-chromatographic separation of the individual reaction products was performed by a combined FID/WLD method in which three capillary columns were passed through

FID: HP-Innowax®, 0.32 mm inner bore, 60 m long, 0.25 μm layer thickness. (FID means flame ionization dector)

WLD: Connection one behind the other of:

HP-plot® Q, 0.32 mm inner bore, 30 m long, 20 μm layer thickness

HP-plot® molecular sieve 5 A, 0.32 mm inner bore, 30 m long, 12 μm layer thickness. (WLD means heat conductivity detector)

The reaction was carried out for a period of two hours. Thereafter, in the reactor system with the alternating layers of catalyst and adsorbent, 60% more propylene oxide was found on the zeolite layers during the thermogravimetric analysis than in the two-layer system.

As anticipated, propylene oxide no longer appeared at the outlet of the reactor. The adsorbed propylene oxide was liberated again on the raising of the temperature.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of epoxides comprising
(1) oxidizing
(a) one or more hydrocarbons in the presence of
(b) oxygen,
(c) at least one reducing agent, and
(d) a catalyst, and
(2) passing the reaction mixture through an arrangement comprising at least one catalyst-containing layer and at least one adsorbent-containing layer which adsorbs the epoxide, wherein, if there is more than one catalyst-containing layer or more than one adsorbent-containing layer, the catalyst-containing layers and the adsorbent-containing layers are arranged alternately one behind the other.

2. The process of claim 1 for the production of epoxides comprising
(1) oxidizing
(a) one or more hydrocarbons in the presence of
(b) oxygen,
(c) at least one reducing agent, and
(b) a catalyst, and
(2) passing the reaction mixture through an arrangement comprising at least two catalyst-containing layer and at least one adsorbent-containing layer which adsorbs the epoxide, wherein, if there is more than one catalyst-containing layer or more than one adsorbent-containing layer, the catalyst-containing layers and the adsorbent-containing layers are arranged alternately one behind the other.

3. The process of claim 1, wherein the arrangement through which the reaction mixture passes comprises a plurality of adsorbent-containing layers arranged in parallel behind each catalyst-containing layer, which may be used alternately for adsorbing and desorbing the reaction product.

4. The process of claim 1, wherein there are from 2 to 10 adsorbent-containing layers arranged in parallel behind each catalyst-containing layer.

5. The process of claim 1, wherein there are from 2 to 5 adsorbent-containing layers arranged in parallel behind each catalyst-containing layer.

6. The process of claim 1, wherein only partial oxidation of the hydrocarbon in the reaction mixture occurs in each of the catalyst-containing layers.

7. The process of claim 1, wherein the catalyst in each catalyst-containing layer comprises a metal catalyst on a metal oxide support, said metal catalyst is selected from the group consisting of cobalt, ruthenium, iridium, nickel, palladium, platinum, copper, silver, gold and mixtures thereof comprising 2 or more of said metals.

8. The process of claim 1, wherein the adsorbent in the adsorbent containing layer comprises hydrophobic zeolites.

9. The process of claim 1, wherein said hydrocarbon comprises an alkene.

10. The process of claim 9, wherein said alkene is selected from the group consisting of ethene, propene and butene.

11. The process according of claim 1, wherein said reducing agent comprises hydrogen, carbon monoxide, or a mixture thereof.

12. The process of claim 1, wherein said oxidation step is carried out at temperatures of 20 to 400° C.
